(19) United States
(12) Patent Application Publication
(10) Pub. No.: US 2020/0002249 A1
(43) Pub. Date: Jan. 2, 2020

(54) METHOD FOR PRODUCING UNSATURATED HYDROCARBON

(71) Applicants: JXTG Nippon Oil & Energy Corporation, Tokyo (JP); National University Corporation Saitama University, Saitama (JP)

(72) Inventors: Hideki KUROKAWA, Saitama (JP); Tatsuya ICHIJO, Tokyo (JP); Nobuhiro KIMURA, Tokyo (JP)

(73) Assignees: JXTG Nippon Oil & Energy Corporation, Tokyo (JP); National University Corporation Saitama University, Saitama (JP)

(21) Appl. No.: 16/485,017

(22) PCT Filed: Dec. 27, 2017
(86) PCT No.: PCT/JP2017/046970

§ 371 Date: Aug. 9, 2019

(30) Foreign Application Priority Data
Feb. 21, 2017 (JP) 2017-030167

(51) Int. Cl. C07C 5/333; B01J 23/02; B01J 23/62; B01J 37/02; B01J 37/08

(52) U.S. Cl. CPC C07C 5/3337; B01J 23/02; B01J 23/626; B01J 37/0205; B01J 37/0207; B01J 37/0236; C07C 2521/02; B01J 37/024; C07C 2527/25; C07C 2523/14; C07C 2523/42; C07C 2523/62; C07C 2523/02; B01J 37/088

(57) ABSTRACT

A method for producing an unsaturated hydrocarbon, comprising: a step of contacting a raw material gas containing an alkane with a dehydrogenation catalyst to obtain a product gas containing at least one unsaturated hydrocarbon selected from a group consisting of olefins and conjugated dienes, wherein the dehydrogenation catalyst contains at least one additive element selected from the group consisting of Na, K, and Ca, Al, Mg, a group 14 metal element, and Pt, and a content of the additive element is 0.05% by mass or more and 0.70% by mass or less based on a total mass of the dehydrogenation catalyst.

### TECHNICAL FIELD

The present invention relates to a method for producing an unsaturated hydrocarbon.

### BACKGROUND ART

An increase in the demand of unsaturated hydrocarbons including butadiene as a raw material for synthetic rubbers, or the like has been anticipated because of motorization centering on Asia in recent years. For example, a method for producing a conjugated diene by a direct dehydrogenation reaction of n-butane using a dehydrogenation catalyst (Patent Literature 1) and methods for producing a conjugated diene by an oxidative dehydrogenation reaction of n-butene (Patent Literatures 2 to 4) have been known as methods for producing butadiene.

### CITATION LIST

### Patent Literature

Patent Literature 1 Japanese Unexamined Patent Publication No. 2014-205135

Patent Literature 2 Japanese Unexamined Patent Publication No. S57-140730

Patent Literature 3 Japanese Unexamined Patent Publication No. S60-1139

Patent Literature 4 Japanese Unexamined Patent Publication No. 2003-220335

### SUMMARY OF INVENTION

### Technical Problem

Along with an increase in the demand of unsaturated hydrocarbons, development of various methods for producing unsaturated hydrocarbons is required, the methods differing in features such as required properties, operating cost, and reaction efficiency of a producing device.

It is an object of the present invention to provide, as a novel method for producing an unsaturated hydrocarbon, a production method in which deposition of coke on a catalyst is little, which can maintain good reaction efficiency for a long time, and which can obtain an unsaturated hydrocarbon with excellent production efficiency.

### Solution to Problem

An aspect of the present invention relates to a method for producing an unsaturated hydrocarbon comprising a step of contacting a raw material gas containing an alkane with a dehydrogenation catalyst to obtain a product gas containing at least one unsaturated hydrocarbon selected from the group consisting of olefins and conjugated dienes, wherein the dehydrogenation catalyst contains at least one additive element selected from the group consisting of Na, K, and Ca, Al, Mg, a group 14 metal element, and Pt, and the content of the additive element is 0.05% by mass or more and 0.70% by mass or less based on the total mass of the dehydrogenation catalyst.

In one aspect, the content of the additive element in the dehydrogenation catalyst may be 0.08% by mass or more and 0.35% by mass or less based on the total mass of the dehydrogenation catalyst.

In one aspect, the molar ratio of Mg to Al in the dehydrogenation catalyst may be 0.30 or more and 0.60 or less.

In one aspect, the molar ratio of the group 14 metal element to Pt in the dehydrogenation catalyst may be 10 or less.

In one aspect, the group 14 metal element may include Sn.

In one aspect, the alkane may be an alkane having 4 to 10 carbon atoms.

In one aspect, the alkane may be butane, the olefin may be butene, and the conjugated diene may be butadiene.

### Advantageous Effects of Invention

According to the present invention, as a novel method for producing an unsaturated hydrocarbon, provided is a production method in which deposition of coke on a catalyst is little, which can maintain good reaction efficiency for a long time, and which can obtain an unsaturated hydrocarbon with excellent production efficiency.

### DESCRIPTION OF EMBODIMENTS

Hereinbelow, one suitable embodiment of the present invention will be described.

A production method of the present embodiment comprises a step of contacting a raw material gas containing an alkane with a dehydrogenation catalyst to obtain a product gas containing at least one unsaturated hydrocarbon selected from the group consisting of olefins and conjugated dienes (hereinbelow, the step is also referred to as the “dehydrogenation step”). In the present embodiment, the dehydrogenation catalyst contains at least one additive element selected from the group consisting of Na, K, and Ca, Al, Mg, a group 14 metal element, and Pt, and the content of the additive element is 0.05% by mass or more and 0.70% by mass or less based on the total mass of the dehydrogenation catalyst.

According to the production method of the present embodiment, by use of a dehydrogenation catalyst containing specific metal elements, it is possible to reduce the amount of coke to be deposited on the catalyst, to maintain good reaction efficiency for a long time, and to obtain an unsaturated hydrocarbon with excellent production efficiency. Although the reason for exerting such effects is not necessarily clear, the reason is assumed as follows.

In the present embodiment, it is considered that high catalyst activity is obtained because the dehydrogenation catalyst contains Al, Mg, the group 14 metal element, and Pt. More specifically, in the dehydrogenation catalyst according to the present embodiment, acid points derived from Al are covered with Mg and the group 14 metal element to weaken the acid property, and thereby, side reactions such as a cracking reaction of alkane are suppressed. Additionally, it is considered that the group 14 metal element and Pt in the dehydrogenation catalyst form bimetallic particles to suppress aggregation of Pt particles with one another as well as to cause electron donation from the group 14 metal element to Pt. It is considered that because of this, dehydrogenation activity is enhanced. Furthermore, it is considered that Pt atoms are diluted in the above bimetallic particles and the Pt atoms act on one molecule of the hydrocarbon at multiple points to thereby suppress the cleavage reaction of a C—C bond. It is considered that a high conversion rate of the alkane and a high reaction selectivity are achieved due to these reasons in the present embodiment.

Additionally, in the present embodiment, the dehydrogenation catalyst contains at least one additive element selected from the group consisting of Na, K, and Ca, and thus, the amount of coke deposited is reduced while the excellent catalyst activity mentioned above is sufficiently maintained. It is considered that this is because acid points derived from Al that were not covered completely with Mg and the group 14 metal element are covered with the additive element to thereby suppress generation of coke caused by the acid points.

In the present embodiment, the raw material gas contains an alkane. The number of carbon atoms of the alkane may be the same as the number of carbon atoms of an intended unsaturated hydrocarbon. The number of carbon atoms of the alkane, for example, may be 4 to 10 or may be 4 to 6.

The alkane may be, for example, chain-like, or may be cyclic. Chain-like alkanes include linear alkanes and branched alkanes. Examples of the chain-like alkane include butane, pentane, hexane, heptane, octane, and decane. More specific examples of the linear alkane include n-butane, n-pentane, n-hexane, n-heptane, n-octane, and n-decane. Additionally, examples of the branched alkane include isobutane, isopentane, 2-methylpentane, 3-methylpentane, 2,3-dimethylpentane, isoheptane, isooctane, and isodecane. Examples of the cyclic alkane include cyclopentane, cyclohexane, cycloheptane, cyclooctane, cyclodecane, and methylcyclohexane. The raw material gas may contain one alkane or may contain two or more alkanes.

In the raw material gas, the partial pressure of the alkane may be 1.0 MPa or less, may be 0.1 MPa or less, or may be 0.01 MPa or less. The conversion rate of the alkane is more likely to be enhanced by lowering the alkane partial pressure of the raw material gas.

Also, the partial pressure of the alkane in the raw material gas is preferably 0.001 MPa or more, more preferably 0.005 MPa or more, from the viewpoint of reducing the size of a reactor with respect to a raw material flow rate.

The raw material gas may further contain an inert gas such as nitrogen or argon. Also, the raw material gas may further contain steam.

When the raw material gas contains steam, the content of the steam is preferably 1.0 times moles or more, more preferably 1.5 times moles or more, with respect to the alkane. Deterioration in the activity of the catalyst may be more markedly suppressed by incorporation of steam in the raw material gas. The content of the steam, for example, may be 50 times moles or less and is preferably 10 times moles or less with respect to the alkane.

The raw material gas may further contain other components such as hydrogen, oxygen, carbon monoxide, carbon dioxide, olefins, and dienes in addition to the above.

In the present embodiment, the product gas contains at least one unsaturated hydrocarbon selected from the group consisting of olefins and conjugated dienes. The number of carbon atoms of each of the olefin and the conjugated diene may be the same as the number of carbon atoms of the alkane, and, for example, may be 4 to 10 or may be 4 to 6.

Examples of the olefin include butene, pentene, hexene, heptene, octene, nonene, and decene, and these may be any isomers. Examples of the conjugated diene include butadiene (1,3-butadiene), 1,3-pentadiene, isoprene, 1,3-hexadiene, 1,3-heptadiene, 1,3-octadiene, 1,3-nonadiene, and 1,3-decadiene. The product gas may be one that contains one unsaturated hydrocarbon or may be one that contains two or more unsaturated hydrocarbons. For example, the product gas may contain an olefin and a conjugated diene.

The production method of the present embodiment can be particularly suitably used for a method that uses a raw material gas containing butane as an alkane among the above, that is, a method for producing at least one unsaturated hydrocarbon selected from the group consisting of butene and butadiene. The butane used for producing at least one unsaturated hydrocarbon selected from the group consisting of butene and butadiene may be n-butane or isobutane. The butane may be a mixture of n-butane and isobutane.

Hereinbelow, the dehydrogenation catalyst in the present embodiment will be described in detail.

The dehydrogenation catalyst is a solid catalyst for catalyzing the dehydrogenation reaction of an alkane, the catalyst containing at least one additive element selected from the group consisting of Na, K, and Ca, Al, Mg, a group 14 metal element, and Pt. Herein, the group 14 metal element means a metal element belonging to the group 14 in a long-form element periodic table defined by the International Union of Pure and Applied Chemistry (IUPAC).

The group 14 metal element may be at least one selected from the group consisting of germanium (Ge), tin (Sn), and lead (Pb), for example. When the group 14 metal element is Sn among these, the effects mentioned above are exerted further markedly.

In the dehydrogenation catalyst, the content of the additive element is 0.05% by mass or more, preferably 0.07% by mass or more, more preferably 0.1% by mass or more, further preferably 0.08% by mass or more, based on the total mass of the dehydrogenation catalyst. It is possible to more markedly reduce the amount of coke deposited by raising the content of the additive element. Additionally, the content of the additive element is 0.70% by mass or less, preferably 0.65% by mass or less, more preferably 0.5% by mass or less, further preferably 0.4% by mass or less, still more preferably 0.35% by mass or less, based on the total mass of the dehydrogenation catalyst. The catalyst activity of the dehydrogenation catalyst tends to further increase by lowering the content of the additive element.

In the dehydrogenation catalyst, the content of Al may be 15% by mass or more or may be 25% by mass or more, based on the total mass of the dehydrogenation catalyst. Also, the content of Al may be 40% by mass or less.

In the dehydrogenation catalyst, the content of Mg is preferably 10% by mass or more, more preferably 13% by mass or more, based on the total mass of the dehydrogenation catalyst. The content of Mg is preferably 20% by mass or less, more preferably 18% by mass or less, based on the total mass of the dehydrogenation catalyst.

In the dehydrogenation catalyst, the content of the group 14 metal element is preferably 1% by mass or more, more preferably 2% by mass or more, based on the total mass of the dehydrogenation catalyst. The content of the group 14 metal element is preferably 8% by mass or less, more preferably 6% by mass or less, based on the total mass of the dehydrogenation catalyst

In the dehydrogenation catalyst, the content of Pt is preferably 0.1% by mass or more, more preferably 0.5% by mass or more, based on the total mass of the dehydrogenation catalyst. The content of Pt is preferably 5% by mass or less, more preferably 3% by mass or less, based on the total mass of the dehydrogenation catalyst. When the content of Pt is 0.1% by mass or more, the amount of platinum per catalyst amount increases, and thus, the size of a reactor can be reduced. Alternatively, when the content of Pt is 5% by mass or less, the size of Pt particles to be formed on the catalyst will be a size suitable for the dehydrogenation reaction to thereby extend the surface area of platinum per unit platinum weight, and thus, a more efficient reaction system can be achieved.

In the dehydrogenation catalyst, the molar ratio of the group 14 metal element to Pt (number of moles of the group 14 metal element/number of moles of Pt) is preferably 2 or more, more preferably 4 or more, from the viewpoint that a side reaction is suppressed and the production efficiency of the unsaturated hydrocarbon is further enhanced. Also, the molar ratio of the group 14 metal element to Pt is preferably 12 or less, more preferably 10 or less, from the viewpoint that excessive covering of the Pt particles with the group 14 metal element is prevented and the production efficiency of the unsaturated hydrocarbon is further enhanced.

In the dehydrogenation catalyst, the molar ratio of Mg to Al (number of moles of Mg/number of moles of Al) is preferably 0.30 or more, more preferably 0.40 or more, from the viewpoint that a side reaction is more markedly suppressed and the production efficiency of the unsaturated hydrocarbon is further enhanced. The molar ratio of Mg to Al is preferably 0.60 or less, more preferably 0.55 or less, from the viewpoint that the dispersibility of Pt in the dehydrogenation catalyst is enhanced.

Note that the content of each metal element in the dehydrogenation catalyst can be measured by a method described in the following Examples.

In one suitable aspect, the dehydrogenation catalyst may be a catalyst in which a group 14 metal element, Pt, and an additive element are supported on a carrier containing Al and Mg.

In the present aspect, the carrier may be a carrier containing alumina (Al₂O₃) and magnesium oxide (MgO) or may be a carrier containing a composite oxide of Al and Mg (e.g., MgAl₂O₄). The carrier also may be a carrier containing the above composite oxide and alumina and/or magnesium oxide.

The content of Al in the carrier may be 20% by mass or more or may be 30% by mass or more, based on the total mass of the carrier. The content of Al in the carrier also may be may be 60% by mass or less or may be 50% by mass or less, based on the total mass of the carrier.

In the carrier, the content of Al in terms of oxide (Al₂O₃) may be 50% by mass or more or may be 60% by mass or more, based on the total mass of the carrier. Also, the content of Al in terms of oxide may be 90% by mass or less or may be 85% by mass or less, based on the total mass of the carrier.

The content of Mg in the carrier may be 5% by mass or more or may be 10% by mass or more, based on the total mass of the carrier. Also, the content of Mg in the carrier may be 30% by mass or less or may be 20% by mass or less, based on the total mass of the carrier.

In the carrier, the content of Mg in terms of oxide (MgO) may be 10% by mass or more or may be 15% by mass or more, based on the total mass of the carrier. Also, the content of Mg in terms of oxide may be 50% by mass or less or may be 35% by mass or less, based on the total mass of the carrier.

In the carrier, the total content of Al and Mg in terms of oxide may be 50% by mass or more, may be 70% by mass or more, or may be 100% by mass, based on the total mass of the carrier.

When the carrier contains a composite oxide of Al and Mg, the content may be 60% by mass or more or may be 80% by mass or more, based on the total mass of the carrier. Also, the content of the composite oxide may be 100% by mass or less or may be 90% by mass or less, based on the total mass of the carrier.

The carrier may further contain a metal element other than Al and Mg. The other metal element may exist as an oxide or may exist as a composite oxide of the metal element and at least one selected from the group consisting of Al and Mg.

The acidity of the carrier is preferably near neutrality from the viewpoint of suppressing a side reaction. Here, the standard over the acidity of the carrier is generally distinguished by the pH in a state where the carrier is dispersed in water. That is, herein, the acidity of the carrier can be represented by the pH of a suspension in which 1% by mass of the carrier is suspended in water. The acidity of the carrier is preferably a pH of 5.0 to 9.0, more preferably a pH of 6.0 to 8.0.

The specific surface area of the carrier may be 50 m²/g or more, for example, and is preferably 80 m²/g or more. Thereby, the dispersibility of Pt to be supported tends to be enhanced. Also, the specific surface area of the carrier may be 300 m²/g or less, for example, and is preferably 200 m²/g or less. In a carrier having such a specific surface area, micropores likely to collapse during firing tends to be low in number, and the dispersibility of Pt to be supported is likely to be enhanced. Note that the specific surface area of the carrier is measured with a BET specific surface area meter using a nitrogen adsorption method.

A method for preparing the carrier is not particularly limited and may be, for example, a sol-gel method, coprecipitation method, hydrothermal synthesis method, impregnation method, or solid-phase synthesis method.

As an example of the method for preparing the carrier, an example of the impregnation method will be shown below. To begin with, to a solution prepared by dissolving a compound containing a first metal element (e.g., Mg), precursor containing a second metal element (e.g., Al) is added, and the solution is stirred. Then, the solvent is removed under reduced pressure, and the resulting solid is dried. The dried solid is fired to thereby obtain a carrier containing the first metal element and the second metal element. In this aspect, the content of the intended metal element contained in the carrier can be adjusted by the concentration of the intended metal element in the solution containing the metal element, the amount of the solution used, and the like.

The compound containing the first metal element may be, for example, a salt or complex containing the first metal element. The salt containing the first metal element may be, for example, an inorganic salt, organic acid salt, or hydrate thereof. The inorganic salt may be, for example, a sulfate, nitrate, chloride, phosphate, or carbonate. The organic salt may be, for example, an acetate or succinate. The complex containing the first metal element may be, for example, an alkoxide complex or ammine complex.

The solvent for dissolving the compound containing the first metal element may be one that can dissolve the compound and be removed under reduced pressure. Examples of the solvent include hydrochloric acid, nitric acid, ammonia water, ethanol, chloroform, and acetone.

Examples of the carrier precursor include the second metal element include alumina (e.g., γ-alumina). The carrier precursor can be prepared by, for example, a sol-gel method, coprecipitation method, or hydrothermal synthesis method. As the carrier precursor, commercially available alumina may be used.

As conditions during stirring, for example, a stirring temperature can be set to 0 to 60° C., and a stirring time can be set to 10 minutes to 24 hours. As conditions during drying, for example, a drying temperature can be set to 100 to 250° C., and a drying time can be set to 3 hours to 24 hours.

Firing can be, for example, performed under an air atmosphere or oxygen atmosphere. Firing may be performed in one stage or multiple stages such as two stages or more. A firing temperature may be a temperature at which a metal precursor can be decomposed, and may be, for example, 200 to 1000° C. or may be 400 to 800° C. Note that, when firing in multiple stages is performed, at least one stage thereof is only required to be at the above firing temperature. A firing temperature in the other stages may be, for example, within the same range as the above and may be 100 to 200° C.

In the dehydrogenation catalyst, a supported metal containing a group 14 metal element, Pt, and an additive element is supported on a carrier. The supported metal may be supported as an oxide or composite oxide on a carrier or may be supported as a single metal on a carrier.

On the carrier, a metal element other than the group 14 metal element, Pt, and the additive element may be further supported. The other metal element may be supported as a single metal on the carrier, may be supported as an oxide, or may be supported as a composite oxide of the other metal element and at least one selected from the group consisting of a group 14 metal element, Pt, and an additive element.

The amount of the group 14 metal element to be supported on the carrier is preferably 1 part by mass or more, more preferably 2 parts by mass or more, with respect to 100 parts by mass of the carrier. Also, the amount of the group 14 metal element to be supported on the carrier may be 9 parts by mass or less or may be 7 parts by mass or less, with respect to 100 parts by mass of the carrier. When the amount of the group 14 metal element is within the above range, catalyst deterioration is further suppressed, and thus, high activity tends to be maintained over a longer period of time.

The amount of Pt to be supported on the carrier is preferably 0.1 parts by mass or more, more preferably 0.5 parts by mass, with respect to 100 parts by mass of the carrier. The amount of Pt to be supported on the carrier may be 5 parts by mass or less or may be 3 parts by mass or less, with respect to 100 parts by mass of the carrier. With such an amount of Pt, Pt particles formed on the catalyst become in a size suitable for the dehydrogenation reaction to enlarge the surface area of platinum per unit platinum weight, and thus, a more efficient reaction system can be achieved. Additionally, with such an amount of Pt, high activity can be maintained over a longer period of time while catalyst cost is suppressed.

The amount of the additive element to be supported on the carrier is preferably 0.05 parts by mass or more, more preferably 0.1 parts by mass or more, further preferably 0.08 parts by mass or more, with respect to 100 parts by mass of the carrier. Thereby acid points derived from Al can be efficiently covered, and the amount of coke deposited can be suppressed more markedly. Also, the amount of the additive element to be supported on the carrier is preferably 0.70 parts by mass or less, more preferably 0.65 parts by mass or less, further more preferably 0.5 parts by mass or less, still more preferably 0.35 parts by mass or less, with respect to 100 parts by mass of the carrier. Thereby, the catalyst activity of the dehydrogenation catalyst is maintained sufficiently high.

The amount of the other metal element to be supported on the carrier, for example, may be 10 parts by mass or less, may be 5 parts by mass or less, or may be 0 parts by mass, with respect to 100 parts by mass of the carrier.

Examples of a method for supporting the metal on the carrier include, but not particularly limited to, an impregnation method, precipitation method, coprecipitation method, kneading method, ionic exchange method, and pore-filling method. In the present aspect, a plurality of supported metals may be supported one by one on the carrier, or a plurality of supported metals may be supported simultaneously on the carrier.

In the present aspect, for example, a dehydrogenation catalyst may be obtained by further supporting an additive element on a catalyst precursor prepared by supporting a group 14 metal element and a Pt on the carrier. Note that, on preparing the catalyst precursor, after the group 14 metal element is supported on the carrier, Pt may be further supported thereon, or after Pt is supported on the carrier, the group 14 metal element may be further supported thereon. Alternatively, the group 14 metal element and Pt may be supported simultaneously on the carrier.

One aspect of a supporting method on the carrier will be shown below. First, a carrier is added to a solution prepared by dissolving a precursor of a supported metal therein, and the solution is stirred. Then, the solvent is removed under reduced pressure, and the resulting solid is dried. The supported metal can be supported on the carrier by firing the dried solid.

In the above supporting method, the precursor of the supported metal may be, for example, a salt or complex containing the supported metal. The salt containing the supported metal may be, for example, an inorganic salt, organic acid salt, or hydrate thereof. The inorganic salt may be, for example, a sulfate, nitrate, chloride, phosphate, or carbonate. The organic salt may be, for example, an acetate or succinate. The complex containing the supported metal may be, for example, an alkoxide complex, or ammine complex.

The solvent for dissolving the precursor of the supported metal may be one that can dissolve the precursor and can be removed under reduced pressure. Examples of the solvent include water, ethanol, and acetone.

In the above supporting method, as conditions during stirring, for example, a stirring temperature can be set to 0 to 60° C., and a stirring time can be set to 10 minutes to 24 hours. As conditions during drying, for example, a drying temperature can be set to 100 to 250° C., and a drying time can be set to 3 hours to 24 hours.

In the above supporting method, firing can be, for example, performed under an air atmosphere or oxygen atmosphere. Firing may be performed in one stage or multiple stages such as two stages or more. A firing temperature may be a temperature at which the precursor of the supported metal precursor can be decomposed. The firing temperature, for example, may be 200 to 1000° C. or may be 400 to 800° C. Note that, when firing in multiple stages is performed, at least one stage thereof is only required to be at the above firing temperature. A firing temperature in the other stages may be, for example, within the same range as the above, and may be 100 to 200° C.

The degree of dispersion of Pt in the dehydrogenation catalyst may be, for example, 10% or more or may be preferably 15% or more. According to the dehydrogenation catalyst having such a degree of dispersion of Pt, high activity tends to be maintained over a longer period of time. Note that the degree of dispersion of Pt indicates a value measured by a method for measuring the degree of dispersion of a metal using CO as adsorption species. Specifically, the degree of dispersion is measured by the following device under the following conditions.

Device: device for measuring degree of dispersion of metal R-6011 manufactured by Ohkura Riken Co., LTD.

Gas flow rate: 30 mL/minute (helium, hydrogen)

Amount of sample: about 0.1 g (precisely measured to four decimal places)

Pretreatment: The temperature is raised to 400° C. over 1 hour under a hydrogen stream to perform a reduction treatment at 400° C. for 60 minutes. Thereafter, the gas is changed from hydrogen to helium to purge the hydrogen at 400° C. for 30 minutes, and cooling to room temperature is then performed under a helium stream. After a detector is stabilized at room temperature, CO pulsing is performed.

Measurement conditions: Carbon monoxide is pulse-injected by 0.0929 cm³ at room temperature (27° C.) under a stream of normal pressure helium gas to measure the amount of adsorption thereof. The adsorption was performed a number of times until the adsorption is saturated (at least 3 times, at most 15 times).

The dehydrogenation catalyst may be molded by a method such as an extrusion method and tablet compression method.

The dehydrogenation catalyst may contain a molding auxiliary agent in the range not to impair the physical properties and catalytic performance of the catalyst from the viewpoint of improving moldability in a molding step. The molding auxiliary agent may be, for example, at least one selected from the group consisting of a thickener, surfactant, humectant, plasticizer, and binder raw material. The molding step of molding the dehydrogenation catalyst may be performed at a suitable stage during the producing step of the dehydrogenation catalyst in consideration of the reactivity of the molding auxiliary agent.

The shape of the molded dehydrogenation catalyst is not particularly limited, and can be appropriately selected according to a form for using the catalyst. For example, the shape of the dehydrogenation catalyst may be a shape such as a pellet shape, granular shape, honeycomb shape, or sponge shape.

As the dehydrogenation catalyst, one subjected to a reduction treatment as a pretreatment may be used. The reduction treatment can be, for example performed by retaining the dehydrogenation catalyst under reducing gas atmosphere at 40 to 600° C. The retention time may be, for example, 0.05 to 24 hours. The reducing gas may be, for example, hydrogen or carbon monoxide.

The induction period at an initial stage of a dehydrogenation reaction can be shortened by using the dehydrogenation catalyst subjected to the reduction treatment. The induction period at the initial stage of the reaction refers to a state where there are very few active metals that have been reduced and activated, among active metals contained in the catalyst, and the activity of the catalyst is low.

Next, the dehydrogenation step in the present embodiment will be described in detail.

The dehydrogenation step is a step of performing a dehydrogenation reaction of an alkane by contacting a raw material gas with a dehydrogenation catalyst to obtain a product gas containing an unsaturated hydrocarbon of alkane.

The dehydrogenation step may be, for example, performed using a reactor filled with the dehydrogenation catalyst, by circulating the raw material gas through the reactor. As the reactor, various reactors used for a gas phase reaction by use of a solid catalyst can be used. Examples of the reactor include a fixed-bed insulation type reactor, radial flow type reactor, and tube-type reactor.

The reaction form of the dehydrogenation reaction may be, for example, a fixed-bed type, moving-bed type, or fluidized-bed type. Among these, a fixed-bed type is preferred from the viewpoint of equipment cost.

The reaction temperature of the dehydrogenation reaction, that is, the temperature in the reactor may be 300 to 800° C., may be 400 to 700° C., or may be 500 to 650° C., from the viewpoint of the reaction efficiency. When the reaction temperature is 300° C. or more, the amount of an unsaturated hydrocarbon to be generated tends to further increase. When the reaction temperature is 800° C. or less, a coking speed is not excessively high, and thus, the high activity of the dehydrogenation catalyst tends to be maintained over a longer period of time.

The reaction pressure, that is, the atmospheric pressure in the reactor may be 0.01 to 1 MPa, may be 0.05 to 0.8 MPa or may be 0.1 to 0.5 MPa. When the reaction pressure is in the above range, the dehydrogenation reaction is likely to proceed, and more excellent reaction efficiency tends to be obtained.

When the dehydrogenation step is performed in a continuous reaction form for continuously supplying a raw material gas, a weight hourly space velocity (hereinbelow, it is referred to as “WHSV”) may be 0.1 h⁻¹ or more or may be 0.5 h⁻¹ or more, for example. The WHSV may also be 20 h⁻¹ or less or may be 10 h⁻¹ or less. Here, the WHSV is a ratio of the supply rate (amount supplied/time) F of the raw material gas to the mass W of the dehydrogenation catalyst (F/W). When the WHSV is 0.1 h⁻¹ or more, the reactor size can be further reduced. When the WHSV is 20 h⁻¹ or less, the conversion rate of the alkane can be further raised. Note that the amounts of the raw material gas and the catalyst to be used may be appropriately selected in a more preferable range according to reaction conditions and the activity of the catalyst, or the like, and the WHSV is not limited to the range.

In the dehydrogenation step, the reactor may be further filled with a catalyst other than the dehydrogenation catalyst described above (hereinbelow, it may be also referred to as the “first dehydrogenation catalyst”).

For example, in the present embodiment, the side downstream of the first dehydrogenation catalyst of the reactor may be further filled with a solid catalyst that catalysts a dehydrogenation reaction from an olefin to a conjugated diene (hereinbelow, it may be also referred to as the “second dehydrogenation catalyst”). The first dehydrogenation catalyst is particularly excellent in reaction activity of a dehydrogenation reaction from an alkane to an olefin, and thus, the proportion of the conjugated diene in a production gas to be obtained can be raised by filling the side downstream of the first dehydrogenation catalyst with the second dehydrogenation catalyst.

The production method according to the present embodiment may further include a step (second step) of performing a dehydrogenation reaction of the olefin by contacting the production gas containing the olefin (first product gas) obtained in the above dehydrogenation step (first step) with a second dehydrogenation catalyst to obtain a product gas containing a conjugated diene (second product gas) (second step). According to such a production method, a product gas containing a larger amount of the conjugated diene can be obtained.

As the second dehydrogenation catalyst, any catalyst for a dehydrogenation reaction of an olefin can be used without particular limitation. For example, as the second dehydrogenation catalyst, a Pt/Al₂O₃-based catalyst, which is often used as a catalyst for a simple dehydrogenation reaction, or a Bi—Mo-based catalyst, which is often used as a catalyst for an oxidative dehydrogenation reaction, can be used.

As described above, according to the production method of the present embodiment, an unsaturated hydrocarbon can be efficiently produced from an alkane while deposition of coke on the catalyst is suppressed. Thus, according to the production method of the present embodiment, the frequency of catalyst reproduction can be lowered. For such a reason, the production method of the present embodiment is very useful when an unsaturated hydrocarbon (particularly, butene and butadiene) is industrially produced.

As described above, a preferred embodiment according to the present invention has been described, but the present invention is not intended to be limited to the embodiment.

### EXAMPLES

Hereinbelow, the present invention will be more specifically described by way of Examples, but the present invention is not intended to be limited to the Examples.

### Example A-1

### <Preparation of Carrier>

A solution prepared by dissolving 18.8 g of Mg(NO₃)₂.6H₂O in 56 mL of water was added to 15 g of alumina classified to 0.5 to 1 mm (Neobead GB-13, manufactured by Mizusawa Industrial Chemicals, Ltd., pH of a suspension obtained by suspending the alumina at a concentration of 1% by mass: 7.9). The obtained mixed solution was stirred at 40° C. and 0.015 MPaA for 30 minutes and at 40° C. and normal pressure for 30 minutes, using a rotary evaporator. Then, water was removed under reduced pressure while the mixed solution was stirred. The obtained solid was dried in an oven at 130° C. overnight. Next, the dried solid was fired under an air flow at 550° C. for 3 hours and at 800° C. for 3 hours. A solution prepared by dissolving 18.8 g of Mg(NO₃)₂.6H₂O in 56 mL of water was again added to the obtained solid, and the same procedure was repeated to obtain a carrier A-1.

<Preparation of Catalyst>

Mixed were 3.0 g of the carrier A-1 and an aqueous solution prepared by dissolving 79.6 mg of H₂PtCl₆.2H₂O in 16 mL of water. The obtained mixed solution was stirred at 40° C. and 0.015 MPaA for 30 minutes and at 40° C. and normal pressure for 30 minutes, using a rotary evaporator. Then, water was removed under reduced pressure while the mixed solution was stirred. The obtained solid was dried in an oven at 130° C. overnight. Next, the dried solid was fired under an air flow at 550° C. for 3 hours. Then, the obtained fired product and a solution prepared by dissolving 0.277 g of SnCl₂.2H₂O in 20 mL of EtOH were mixed. The obtained mixed solution was stirred at 40° C. under normal pressure for an hour using a rotary evaporator, and then, EtOH was removed under reduced pressure. The obtained solid was dried in an oven at 130° C. overnight. Next, the dried solid was fired under an air flow at 550° C. for 3 hours. Subsequently, the obtained fired product and a solution prepared by dissolving 54.5 mg of Ca(NO₃)₂.4H₂O in 5 mL of water were mixed. The obtained mixed solution was stirred at 40° C. and 0.015 MPaA for 30 minutes and at 40° C. and normal pressure for 30 minutes, using a rotary evaporator. Then, water was removed under reduced pressure while the mixed solution was stirred. The obtained solid was dried in an oven at 130° C. overnight. Next, after the dried solid was fired under an air flow at 550° C. for 3 hours, reduction by hydrogen was performed to obtain a catalyst A-1. The reduction by hydrogen was performed by retaining the fired solid under a flow of a mixed gas prepared by mixing hydrogen and nitrogen at 1:1 (molar ratio) at 550° C. for 3 hours.

The content of Pt, the content of Sn, the content of Mg, the content of Al, and the content of the additive element (Ca) in the obtained catalyst A-1 were measured with an X-ray fluorescence analysis method (XRF). The X-ray fluorescence analysis method was performed using a measuring device PW2400 (manufactured by PANalytical), and quantification of the contents was performed using standardless quantitative calculation program UniQuant4. Preparation of a measurement sample for XRF was performed as follows. After 125 mg of a sample (for example, the catalyst A-1) and 125 mg of cellulose (binder) were measured in an agate mortar and mixed for 15 minutes, the mixture was put into a 20-mmϕ tablet molding machine and pressure-molded under conditions of 300 kgf·cm² for 10 minutes.

As a result of measurement, in the catalyst A-1, the content of Pt was 1% by mass, the content of Sn was 4.9% by mass, the content of Mg was 16% by mass, the content of Al was 36% by mass, the content of Ca as the additive element was 0.3% by mass, and the molar ratio of Sn to Pt (Sn/Pt) was 8.

<Production of Unsaturated Hydrocarbon>

A tube-type reactor was filled with 0.5 g of the catalyst A-1, and the reactor was connected to a fixed-bed circulation type reaction device. Next, while a mixed gas of hydrogen and He (hydrogen:He=4:6 (mole ratio)) was circulated at 50 mL/min, the temperature of the reactor was raised to 550° C., and the reactor was retained at the temperature for an hour. Then, a mixed gas of He and water was supplied to the reactor and retained for 30 minutes to perform steaming of the catalyst. Here, the molar ratio between He and water in the mixed gas was adjusted to 4:3. The supply rate of the mixed gas to the reactor was adjusted to 87 mL/min. Subsequently, a mixed gas of n-butane, He, and water (raw material gas) was supplied to the reactor, and dehydrogenation reaction on n-butane in the raw material gas was performed. Here, the molar ratio of n-butane, He, and water in the raw material gas was adjusted to 1:4:3. The supply rate of the raw material gas to the reactor was adjusted to 99 mL/min. The WHSV was adjusted to 3.8 h⁻¹. The pressure of the raw material gas in the reactor was adjusted to atmospheric pressure.

At a point of time when 20 minutes elapsed from the start of the reaction, a product material (product gas) of the dehydrogenation reaction was collected from the tube-type reactor. Additionally, at a point of time when 360 minutes elapsed from the start of the reaction, a product material (product gas) of the dehydrogenation reaction was collected from the tube-type reactor. Note that the start of the reaction is the time at which the supply of the raw material gas was started. The product gas collected at each time point was analyzed using a gas chromatograph (TCD-GC) equipped with a thermal conductivity detector. As a result of analysis, the product gas was confirmed to contain n-butene (1-butene, t-2-butene, and c-2-butene) and 1,3-butadiene. The concentration of n-butane (unit: % by mass), the concentration of n-butene (unit: % by mass), and the concentration of 1,3-butadiene (unit: % by mass) in the product gas collected at each time point were quantified based on the above gas chromatograph.

From the concentration of each of n-butane, n-butene, and 1,3-butadiene in the product gas, the conversion rate of the raw material (conversion rate of n-butane) and the selectivity of butene and 1,3-butadiene (selectivity of butene+butadiene) were calculated. Note that the conversion rate of n-butane is defined by the following formula (1) and the selectivity of butene+butadiene is defined by the following formula (2).

R_{c}=(1−Mₚ/M₀)×100 (1)

R_{S}=(M_{b}+M_{c})/(M₀−M_{P})×100 (2)

R_{c} in the formula (1) is the conversion rate of n-butane. R_{S} in the formula (2) is the selectivity of butene+butadiene. M₀ in the formulas (1) and (2) is the number of moles of n-butane in the raw material gas. M_{P} in the formula (1) is the number of moles of n-butane in the product gas. M_{b} in the formula (2) is the number of moles of n-butene (1-butene, t-2-butene, and c-2-butene) in the product gas. M_{c} in the formula (2) is the number of moles of 1,3-butadiene in the product gas.

Additionally, at a point of time when 360 minutes elapsed from the start of the reaction, the used catalyst was collected from the tube-type reactor, and the amount of coke deposited on the catalyst (the amount of coke with respect to the total amount of the used catalyst (% by mass)) was measured by a method shown below. Of the order of 20 mg of the used catalyst was placed in a sample holder for a thermogravimetric analysis (TGA) apparatus. After the sample temperature was raised at a heating rate of 50° C. per minute from room temperature to 200° C. in a stream of nitrogen, the temperature was retained for 10 minutes. The weight of the sample at this time is taken as G₁. Next, after the sample temperature was raised at a heating rate of 15° C. per minute from 200° C. to 700° C. in a stream of air, the temperature was retained for 5 minutes. The weight of the sample at this time is taken as G₂. The amount of coke deposited on the catalyst C (unit: % by mass) was determined using the formula (3) shown below.

C=(G₁−G₂)G₂×100 (3)

As a result of analysis, at a point of time when 20 minutes elapsed, the conversion rate of n-butane was 60.2%, and the selectivity of butene+butadiene was 96.4%. At a point of time when 360 minutes elapsed, the conversion rate of n-butene was 51.8%, and the selectivity of butene+butadiene was 96.7%, and the amount of coke at a point of time when 360 minutes elapsed was 0.8% by mass.

### Example A-2

Preparation of a catalyst was performed in the same manner as in Example A-1 to obtain a catalyst A-2 except that the amount of Ca(NO₃)₂.4H₂O to be added was set to 109.0 mg on preparing the catalyst. When the obtained catalyst A-2 was analyzed, the content of Pt was 1% by mass, the content of Sn was 4.9% by mass, the content of Mg was 16% by mass, the content of Al was 35% by mass, the amount of Ca as the additive element was 0.6% by mass, and the molar ratio of Sn to Pt (Sn/Pt) was 8.

Additionally, production of an unsaturated hydrocarbon was performed in the same manner as in Example A-1 except that the catalyst A-2 was used instead of the catalyst A-1, and analysis of the product gas and measurement of the amount of coke were performed. As a result, at a point of time when 20 minutes elapsed, the conversion rate of n-butane was 52.8%, and the selectivity of butene+butadiene was 96.8%. At a point of time when 360 minutes elapsed, the conversion rate of n-butene was 46.1%, and the selectivity of butene+butadiene was 96.8%, and the amount of coke at a point of time when 360 minutes elapsed was 0.6% by mass.

### Example A-3

Preparation of a catalyst was performed in the same manner as in Example A-1 to obtain a catalyst A-3 except that the amount of Ca(NO₃)₂.4H₂O to be added was set to 127.2 mg on preparing the catalyst. When the obtained catalyst A-3 was analyzed, the content of Pt was 1% by mass, the content of Sn was 4.9% by mass, the content of Mg was 16% by mass, the content of Al was 35% by mass, the amount of Ca as the additive element was 0.7% by mass, and the molar ratio of Sn to Pt (Sn/Pt) was 8.

Additionally, production of an unsaturated hydrocarbon was performed in the same manner as in Example A-1 except that the catalyst A-3 was used instead of the catalyst A-1, and analysis of the product gas and measurement of the amount of coke were performed. As a result, at a point of time when 20 minutes elapsed, the conversion rate of n-butane was 48.8%, and the selectivity of butene+butadiene was 96.0%. At a point of time when 360 minutes elapsed, the conversion rate of n-butene was 43.3%, and the selectivity of butene+butadiene was 96.8%, and the amount of coke at a point of time when 360 minutes elapsed was 0.4% by mass.

### Comparative Example a-1

Preparation of a catalyst was performed in the same manner as in Example A-1 to obtain a catalyst a-1 except that Ca was not supported on preparing the catalyst. When the obtained catalyst a-1 was analyzed, the content of Pt was 1% by mass, the content of Sn was 4.9% by mass, the content of Mg was 16% by mass, the content of Al was 36% by mass, and the molar ratio of Sn to Pt (Sn/Pt) was 8.

Additionally, production of an unsaturated hydrocarbon was performed in the same manner as in Example A-1 except that the catalyst a-1 was used instead of the catalyst A-1, and analysis of the product gas and measurement of the amount of coke were performed. As a result, at a point of time when 20 minutes elapsed, the conversion rate of n-butane was 60.6%, and the selectivity of butene+butadiene was 95.9%. At a point of time when 360 minutes elapsed, the conversion rate of n-butene was 49.8%, and the selectivity of butene+butadiene was 96.6%, and the amount of coke at a point of time when 360 minutes elapsed was 1.8% by mass.

### Comparative Example a-2

Preparation of a catalyst was performed in the same manner as in Example A-1 to obtain a catalyst a-2 except that the amount of Ca(NO₃)₂.4H₂O to be added was set to 163.5 mg on preparing the catalyst. When the obtained catalyst a-2 was analyzed, the content of Pt was 1% by mass, the content of Sn was 4.9% by mass, the content of Mg was 16% by mass, the content of Al was 35% by mass, the amount of Ca as the additive element was 0.9% by mass, and the molar ratio of Sn to Pt (Sn/Pt) was 8.

Additionally, production of an unsaturated hydrocarbon was performed in the same manner as in Example A-1 except that the catalyst a-2 was used instead of the catalyst A-1, and analysis of the product gas and measurement of the amount of coke were performed. As a result, at a point of time when 20 minutes elapsed, the conversion rate of n-butane was 41.7%, and the selectivity of butene+butadiene was 96.8%. At a point of time when 360 minutes elapsed, the conversion rate of n-butene was 37.3%, and the selectivity of butene+butadiene was 96.4%, and the amount of coke at a point of time when 360 minutes elapsed was 0.5% by mass.

### Comparative Example a-3

Preparation of a catalyst was performed in the same manner as in Example A-1 to obtain a catalyst a-3 except that the amount of Ca(NO₃)₂.4H₂O to be added was set to 218.0 mg on preparing the catalyst. When the obtained catalyst a-3 was analyzed, the content of Pt was 1% by mass, the content of Sn was 4.9% by mass, the content of Mg was 16% by mass, the content of Al was 35% by mass, the amount of Ca as the additive element was 1.2% by mass, and the molar ratio of Sn to Pt (Sn/Pt) was 8.

Additionally, production of an unsaturated hydrocarbon was performed in the same manner as in Example A-1 except that the catalyst a-3 was used instead of the catalyst A-1, and analysis of the product gas and measurement of the amount of coke were performed. As a result, at a point of time when 20 minutes elapsed, the conversion rate of n-butane was 30.6%, and the selectivity of butene+butadiene was 96.8%. At a point of time when 360 minutes elapsed, the conversion rate of n-butene was 28.9%, and the selectivity of butene+butadiene was 96.8%, and the amount of coke at a point of time when 360 minutes elapsed was 0.8% by mass.

The results of Examples A-1, A-2, and A-3 and Comparative Examples a-1, a-2, and a-3 are shown Table 1.

TABLE 1

| | | Example | Example | Example | Comparative | Comparative | Comparative |
|---|---|---|---|---|---|---|---|
| | | A-1 | A-2 | A-3 | Example a-1 | Example a-2 | Example a-3 |
| Dehydrogenation | Additive element | Ca | Ca | Ca | — | Ca | Ca |
| catalyst | Content of the | 0.3 | 0.6 | 0.7 | — | 0.9 | 1.2 |
| | additive element | | | | | | |
| | (% by mass) | | | | | | |
| Conversion rate | After 20 minutes | 60.2 | 52.8 | 48.8 | 60.6 | 41.7 | 30.6 |
| of n-butane | After 360 minutes | 51.8 | 46.1 | 43.3 | 49.8 | 37.3 | 28.9 |
| Selectivity of | After 20 minutes | 96.4 | 96.8 | 96.0 | 95.9 | 96.8 | 96.8 |
| butene + | After 360 minutes | 96.7 | 96.8 | 96.8 | 96.6 | 96.4 | 96.8 |
| butadine | | | | | | | |
| Amount of coke | After 360 minutes | 0.8 | 0.6 | 0.4 | 1.8 | 0.5 | 0.8 |
| (% by mass) | | | | | | | |

### Example B-1

Preparation of a catalyst was performed in the same manner as in Example A-1 to obtain a catalyst B-1 except that 7.97 mg of K(NO₃)₂ was used instead of Ca(NO₃)₂.4H₂O on preparing the catalyst. When the obtained catalyst B-1 was analyzed, the content of Pt was 1% by mass, the content of Sn was 4.9% by mass, the content of Mg was 16% by mass, the content of Al was 36% by mass, the amount of K as the additive element was 0.1% by mass, and the molar ratio of Sn to Pt (Sn/Pt) was 8.

Additionally, production of an unsaturated hydrocarbon was performed in the same manner as in Example A-1 except that the catalyst B-1 was used instead of the catalyst A-1, and analysis of the product gas and measurement of the amount of coke were performed. As a result, at a point of time when 20 minutes elapsed, the conversion rate of n-butane was 59.3%, and the selectivity of butene+butadiene was 95.8%. At a point of time when 360 minutes elapsed, the conversion rate of n-butene was 48.8%, and the selectivity of butene+butadiene was 96.3%, and the amount of coke at a point of time when 360 minutes elapsed was 1.0% by mass.

### Example B-2

Preparation of a catalyst was performed in the same manner as in Example A-1 to obtain a catalyst B-2 except that 23.91 mg of K(NO₃)₂ was used instead of Ca(NO₃)₂.4H₂O on preparing the catalyst. When the obtained catalyst B-2 was analyzed, the content of Pt was 1% by mass, the content of Sn was 4.9% by mass, the content of Mg was 16% by mass, the content of Al was 36% by mass, the amount of K as the additive element was 0.3% by mass, and the molar ratio of Sn to Pt (Sn/Pt) was 8.

Additionally, production of an unsaturated hydrocarbon was performed in the same manner as in Example A-1 except that the catalyst B-2 was used instead of the catalyst A-1, and analysis of the product gas and measurement of the amount of coke were performed. As a result, at a point of time when 20 minutes elapsed, the conversion rate of n-butane was 52.5%, and the selectivity of butene+butadiene was 95.7%. At a point of time when 360 minutes elapsed, the conversion rate of n-butene was 43.4%, and the selectivity of butene+butadiene was 95.7%, and the amount of coke at a point of time when 360 minutes elapsed was 0.7% by mass.

### Example B-3

Preparation of a catalyst was performed in the same manner as in Example A-1 to obtain a catalyst B-3 except that 31.88 mg of K(NO₃)₂ was used instead of Ca(NO₃)₂.4H₂O on preparing the catalyst. When the obtained catalyst B-3 was analyzed, the content of Pt was 1% by mass, the content of Sn was 4.9% by mass, the content of Mg was 16% by mass, the content of Al was 36% by mass, the amount of K as the additive element was 0.4% by mass, and the molar ratio of Sn to Pt (Sn/Pt) was 8.

Additionally, production of an unsaturated hydrocarbon was performed in the same manner as in Example A-1 except that the catalyst B-3 was used instead of the catalyst A-1, and analysis of the product gas and measurement of the amount of coke were performed. As a result, at a point of time when 20 minutes elapsed, the conversion rate of n-butane was 51.1%, and the selectivity of butene+butadiene was 96.7%. At a point of time when 360 minutes elapsed, the conversion rate of n-butene was 43.5%, and the selectivity of butene+butadiene was 96.5%, and the amount of coke at a point of time when 360 minutes elapsed was 0.8% by mass.

### Comparative Example b-1

Preparation of a catalyst was performed in the same manner as in Example A-1 to obtain a catalyst b-1 except that 63.76 mg of K(NO₃)₂ was used instead of Ca(NO₃)₂.4H₂O on preparing the catalyst. When the obtained catalyst b-1 was analyzed, the content of Pt was 1% by mass, the content of Sn was 4.9% by mass, the content of Mg was 16% by mass, the content of Al was 35% by mass, the amount of K as the additive element was 0.8% by mass, and the molar ratio of Sn to Pt (Sn/Pt) was 8.

Additionally, production of an unsaturated hydrocarbon was performed in the same manner as in Example A-1 except that the catalyst b-1 was used instead of the catalyst A-1, and analysis of the product gas and measurement of the amount of coke were performed. As a result, at a point of time when 20 minutes elapsed, the conversion rate of n-butane was 30.8%, and the selectivity of butene+butadiene was 96.8%. At a point of time when 360 minutes elapsed, the conversion rate of n-butene was 22.7%, and the selectivity of butene+butadiene was 96.0%, and the amount of coke at a point of time when 360 minutes elapsed was 0.5% by mass.

### Comparative Example b-2

Preparation of a catalyst was performed in the same manner as in Example A-1 to obtain a catalyst b-2 except that 310.83 mg of K(NO₃)₂ was used instead of Ca(NO₃)₂.4H₂O on preparing the catalyst. When the obtained catalyst b-2 was analyzed, the content of Pt was 1% by mass, the content of Sn was 4.9% by mass, the content of Mg was 15% by mass, the content of Al was 34% by mass, the amount of K as the additive element was 3.9% by mass, and the molar ratio of Sn to Pt (Sn/Pt) was 8.

Additionally, production of an unsaturated hydrocarbon was performed in the same manner as in Example A-1 except that the catalyst b-2 was used instead of the catalyst A-1, and analysis of the product gas and measurement of the amount of coke were performed. As a result, at a point of time when 20 minutes elapsed, the conversion rate of n-butane was 1.2%, and the selectivity of butene+butadiene was 89.5%. Since the conversion rate was very low, the reaction was stopped here.

The results of Examples B-1 to B-3 and Comparative Examples b-1 to b-2 are shown in Table 2.

TABLE 2

| | | Example | Example | Example | Comparative | Comparative |
|---|---|---|---|---|---|---|
| | | B-1 | B-2 | B-3 | Example b-1 | Example b-2 |
| Dehydrogenation | Additive element | K | K | K | K | K |
| catalyst | Content of the | 0.1 | 0.3 | 0.4 | 0.8 | 3.9 |
| | additive element | | | | | |
| | (% by mass) | | | | | |
| Conversion rate | After 20 minutes | 59.3 | 52.5 | 51.1 | 30.8 | 1.2 |
| of n-butane | After 360 minutes | 48.8 | 43.4 | 43.5 | 22.7 | — |
| Selectivity of | After 20 minutes | 95.8 | 95.7 | 96.7 | 96.8 | 89.5 |
| butene + | After 360 minutes | 96.3 | 95.7 | 96.5 | 96.0 | — |
| butadine | | | | | | |
| Amount of coke | After 360 minutes | 1.0 | 0.7 | 0.8 | 0.5 | — |
| (% by mass) | | | | | | |

### Comparative Example c-1

Preparation of a catalyst was performed in the same manner as in Example A-1 to obtain a catalyst c-1 except that 162.63 mg of Mg(NO₃)₂.6H₂O was used instead of Ca(NO₃)₂.4H₂O on preparing the catalyst. When the obtained catalyst c-1 was analyzed, the content of Pt was 1% by mass, the content of Sn was 4.9% by mass, the content of Mg was 16.5% by mass, the content of Al was 36% by mass, and the molar ratio of Sn to Pt (Sn/Pt) was 8. Note that the amount of Mg of Mg(NO₃)₂.6H₂O added instead of Ca(NO₃)₂.4H₂O was an amount corresponding to 0.5% by mass in the catalyst c-1.

Additionally, production of an unsaturated hydrocarbon was performed in the same manner as in Example A-1 except that the catalyst c-1 was used instead of the catalyst A-1, and analysis of the product gas and measurement of the amount of coke were performed. As a result, at a point of time when 20 minutes elapsed, the conversion rate of n-butane was 59.2%, and the selectivity of butene+butadiene was 95.9%. At a point of time when 360 minutes elapsed, the conversion rate of n-butene was 47.3%, and the selectivity of butene+butadiene was 96.5%, and the amount of coke at a point of time when 360 minutes elapsed was 1.3% by mass.

### Comparative Example c-2

Preparation of a catalyst was performed in the same manner as in Example A-1 to obtain a catalyst c-2 except that 57.67 mg of La(NO₃)₂.6H₂O was used instead of Ca(NO₃)₂.4H₂O on preparing the catalyst. When the obtained catalyst c-2 was analyzed, the content of Pt was 1% by mass, the content of Sn was 4.9% by mass, the content of Mg was 16% by mass, the content of Al was 35% by mass, the amount of La was 0.6% by mass, and the molar ratio of Sn to Pt (Sn/Pt) was 8.

Additionally, production of an unsaturated hydrocarbon was performed in the same manner as in Example A-1 except that the catalyst c-2 was used instead of the catalyst A-1, and analysis of the product gas and measurement of the amount of coke were performed. As a result, at a point of time when 20 minutes elapsed, the conversion rate of n-butane was 60.4%, and the selectivity of butene+butadiene was 95.2%. At a point of time when 360 minutes elapsed, the conversion rate of n-butene was 47.7%, and the selectivity of butene+butadiene was 95.9%/0, and the amount of coke at a point of time when 360 minutes elapsed was 2.0% by mass.

### Comparative Example c-3

Preparation of a catalyst was performed in the same manner as in Example A-1 to obtain a catalyst c-3 except that 162.63 mg of Sr(NO₃)₂ was used instead of Ca(NO₃)₂.4H₂O on preparing the catalyst. When the obtained catalyst c-3 was analyzed, the content of Pt was 1% by mass, the content of Sn was 4.9% by mass, the content of Mg was 16% by mass, the content of Al was 35% by mass, the amount of Sr was 0.6% by mass, and the molar ratio of Sn to Pt (Sn/Pt) was 8.

Additionally, production of an unsaturated hydrocarbon was performed in the same manner as in Example A-1 except that the catalyst c-3 was used instead of the catalyst A-1, and analysis of the product gas and measurement of the amount of coke were performed. As a result, at a point of time when 20 minutes elapsed, the conversion rate of n-butane was 58.1%, and the selectivity of butene+butadiene was 95.9%. At a point of time when 360 minutes elapsed, the conversion rate of n-butene was 48.1%, and the selectivity of butene+butadiene was 96.4%, and the amount of coke at a point of time when 360 minutes elapsed was 1.2% by mass.

The results of Comparative Examples c-1 to c-3 are shown in Table 3.

TABLE 3

| | | Comparative | Comparative | Comparative |
|---|---|---|---|---|
| | | Example c-1 | Example c-2 | Example c-3 |
| Dehydrogenation | Additive element | Mg | La | Sr |
| catalyst | Content of the additive | 0.5 | 0.6 | 0.6 |
| | element (% by mass) | | | |
| Conversion rate of | After 20 minutes | 59.2 | 60.4 | 58.1 |
| n-butane | After 360 minutes | 47.3 | 47.7 | 48.1 |
| Selectivity of butene + | After 20 minutes | 95.9 | 95.2 | 95.9 |
| butadiene | After 360 minutes | 96.5 | 95.9 | 96.4 |
| Amount of coke | After 360 minutes | 1.3 | 2.0 | 1.2 |
| (% by mass) | | | | |

### INDUSTRIAL APPLICABILITY

According to the method for producing an unsaturated hydrocarbon in accordance with the present invention, deposition of coke on a catalyst is little, good reaction efficiency can be maintained for a long time, and an unsaturated hydrocarbon can be obtained with excellent production efficiency.

1. A method for producing an unsaturated hydrocarbon, comprising:
a step of contacting a raw material gas containing an alkane with a dehydrogenation catalyst to obtain a product gas containing at least one unsaturated hydrocarbon selected from a group consisting of olefins and conjugated dienes, wherein
the dehydrogenation catalyst contains at least one additive element selected from a group consisting of Na, K, and Ca, Al, Mg, a group 14 metal element, and Pt, and
a content of the additive element is 0.05% by mass or more and 0.70% by mass or less based on a total mass of the dehydrogenation catalyst.

2. The method according to claim 1, wherein the content of the additive element is 0.08% by mass or more and 0.35% by mass or less, based on the total mass of the dehydrogenation catalyst.

3. The method according to claim 1, wherein a molar ratio of the Mg to the Al is 0.30 or more and 0.60 or less.

4. The method according to claim 1, wherein a molar ratio of the group 14 metal element to the Pt is 10 or less.

5. The method according to claim 1, wherein the group 14 metal element includes Sn.

6. The method according to claim 1, wherein the alkane is an alkane having 4 to 10 carbon atoms.

7. The method according to claim 1, wherein the alkane is butane, the olefin is butene, and the conjugated diene is butadiene.
